(19) United States
(12) Patent Application Publication
(10) Pub. No.: US 2023/0329599 A1
(43) Pub. Date: Oct. 19, 2023

(54) ELECTROCHEMICAL SENSOR BASED ON AN ASSOCIATED SENSOR

(71) Applicants: Min Zhang, Guangdong (CN); Hongyang Cao, Guangdong (CN)

(72) Inventors: Min Zhang, Guangdong (CN); Hongyang Cao, Guangdong (CN)

(21) Appl. No.: 17/972,593

(22) Filed: Oct. 25, 2022

(30) Foreign Application Priority Data
Apr. 14, 2022 (CN) 202220856387.0
Apr. 14, 2022 (CN) 2022208563870

(51) Int. Cl. A61B 5/1468

(52) U.S. Cl. CPC A61B 5/1468; A61B 2562/227

(57) ABSTRACT

The invention relates to an electrochemical sensor based on an associated sensor, including an interface base and a probe part, where the interface base is provided with two groups of electrical connection terminals, the probe part is provided with a working sensor and an associated sensor, both sensors are provided with a counter electrode, a working electrode and a reference electrode, and the counter electrode of the working sensor and the counter electrode of the associated sensor are the same electrode. The invention adds the associated sensor to the probe part, and the two sensors with high similarity share the same counter electrode, and form an associated relationship with an original working sensor; the associated sensor is equivalent to a “standard ruler”, works in a known calibration environment and provides a standard reference value for the working sensor to be corrected, so as to obtain a more accurate result.

### BACKGROUND OF THE INVENTION

The invention relates to the field of electrochemical sensors, in particular to an electrochemical sensor based on an associated sensor.

Existing electrochemical sensors generally use a three-electrode technology, by adding a reference electrode to remove the influence of current changes on the electrochemical reaction bias. Electrochemical sensors are used in many fields, especially in the medical field, which has high requirements on the sensitivity and output accuracy of sensors; otherwise it may cause medical accidents or even endanger the lives of patients.

In the medical field, taking the measurement of human glucose concentration as an example, although the enzyme that mainly controls the sensitivity of the sensors will not decrease with the process of chemical reaction in theory, the enzyme that effectively participates in the chemical reaction is continuously decreasing in practical application, and its activity will also gradually decrease as the environment changes.

The traditional method is to use empirical compensation values, or to measure the attenuation curve and fit it into a function formula, and then to compensate the attenuated part in actual use, so as to obtain output results as close to the real values as possible. This will bring some problems. For example, the empirical compensation values are relatively fixed and cannot reflect the subtle differences in the signal output of each sensor; the measured attenuation curve itself is nonlinear, and errors are constantly introduced in the process of empirical value-fitting-compensation regression, resulting in some errors between the final results and the actual results.

The above problems are worth solving.

### BRIEF SUMMARY OF THE INVENTION

In order to overcome the shortcomings of the prior art, the invention provides an electrochemical sensor based on an associated sensor.

The technical solutions of the invention are as follows:

An electrochemical sensor based on an associated sensor includes
an interface base and a probe part, where the interface base is provided with two groups of electrical connection terminals, the probe part is provided with a working sensor and an associated sensor, both sensors are provided with a counter electrode, a working electrode and a reference electrode, and the counter electrode of the working sensor and the counter electrode of the associated sensor are the same electrode;
one group of electrical connection terminals is correspondingly connected with the counter electrode, working electrode and reference electrode of the working sensor, and the other group of electrical connection terminals is correspondingly connected with the counter electrode, working electrode and reference electrode of the associated sensor.

Further, each group of electrical connection terminals includes a first terminal, a second terminal and a third terminal, where the first terminal is connected with the counter electrode, the second terminal is connected with the working electrode, and the third terminal is connected with the reference electrode.

Further, the associated sensor is arranged at an outer end of the probe part.

Further, the counter electrode is arranged on a back side of the probe part.

Further, the counter electrode extends from the working sensor to the associated sensor.

The invention according to the above solutions has the beneficial effects of:

The invention adds the associated sensor to the probe part, and the two sensors with high similarity share the same counter electrode, and form an associated relationship with an original working sensor; the prediction of the nonlinear attenuation process of the existing sensor is completely abandoned, and the associated sensor is equivalent to a “standard ruler”, works in a known calibration environment and provides a standard reference value for the working sensor to be corrected, so as to obtain a more accurate result.

### BRIEF DESCRIPTION OF THE DRAWINGS

FIG. 1 is a schematic diagram of a front structure of the present invention;

FIG. 2 is a schematic diagram of a rear structure of the present invention.

In the figures, 1. Interface base; 11. First terminal; 12. Second terminal; 13. Third terminal;
2. Probe part; 21. Working sensor; 22. Associated sensor;
201. Counter electrode; 202. Working electrode; 203. Reference electrode.

### DETAILED DESCRIPTION OF THE INVENTION

In order to better understand the purpose, technical solutions and technical effects of the invention, the invention will be further explained below with reference to the accompanying drawings and embodiments. Meanwhile, it is stated that the embodiments described below are only used to explain the present invention rather than limiting the present invention.

It should be noted that when an element is referred to as being “fixed to” or “arranged on” another element, it can be directly on another element or an intervening element may also be present. When an element is referred to as being “connected” to another element, it can be directly connected to another element or an intervening element may also be present.

The directions or positions indicated by the terms “front”, “back”, “top”, “bottom”, etc. are based on the directions or positions shown in the accompanying drawings, which are only for the convenience of description and cannot be understood as restrictions on the technical solutions.

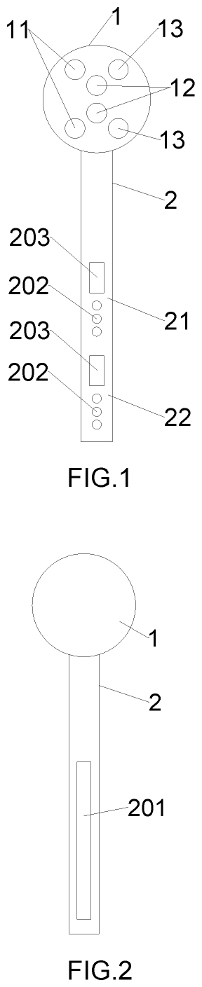

As shown in FIG. 1 and FIG. 2, an electrochemical sensor based on an associated sensor includes an interface base 1 and a probe part 2. The interface base 1 is provided with two groups of electrical connection terminals, the probe part 2 is provided with a working sensor 21 and an associated sensor 22. Both sensors are provided with a counter electrode 201, a working electrode 202 and a reference electrode 203. The counter electrode 201 of the working sensor 21 and the counter electrode 201 of the associated sensor 22 are the same electrode.

In the invention, one group of electrical connection terminals is correspondingly connected with the counter electrode 201, the working electrode 202 and the reference electrode 203 of the working sensor 21, and the other group of electrical connection terminals is correspondingly connected with the counter electrode 201, the working electrode 202 and the reference electrode 203 of the associated sensor 22.

Each group of electrical connection terminals includes a first terminal 11, a second terminal 12 and a third terminal 13. The first terminal 11 and the third terminal 13 are used for power supply, and the second terminal 12 is used for output signal. In electrical connection, the first terminal 11 is connected with the opposite electrode 201, the second terminal 12 is connected with the working electrode 202, and the third terminal 13 is connected with the reference electrode 203. For example, the first terminal 11 of the first group and the first terminal 11 of the second group are connected with the counter electrode 201 at the same time; the second terminal 12 of the first group is connected with the working electrode 202 of the working sensor 21, and the third terminal 13 of the first group is connected with the reference electrode 203 of the working sensor 21; the second terminal 12 of the second group is connected with the working electrode 202 of the associated sensor 22, and the third terminal 13 of the second group is connected with the reference electrode 203 of the associated sensor 22. The specific connection method is to connect the electrical connection terminal with the corresponding electrode through the printed circuit.

In a preferred embodiment, the associated sensor 22 is arranged at an outer end of the probe part 2, separated from the working sensor 21, so that the two sensors can be distinguished and work in two different settings.

The use principle of the invention is: the working sensor 21 works in the environment to be measured; the associated sensor 22 works in a calibration environment, which refers to an environment with standard measurement values, such as a solution with a known glucose concentration or uric acid concentration; the working sensor 21 and the associated sensor 22 work at the same time to measure the current values at respective concentrations; those skilled in the art can obtain the following linear relationship between the solution concentration ratio and the sensor output value (taking the glucose concentration value as an example):

The current value Ix of the working sensor 21/the current value I0 of the associated sensor 22=the glucose concentration value Cx of the working sensor 21/the glucose concentration value C0 of the associated sensor 22;

The current value I0 of the associated sensor 22 and the current value Ix of the working sensor 21 are then measured, and because the glucose concentration value C0 of the associated sensor 22 is known, the glucose concentration value Cx=Ix*C0/I0 of the working sensor 21 can be finally obtained.

It can be seen that the invention can obtain the solution concentration value of the working sensor 21 by calculating the ratio of the output current of the working sensor 21 and the associated sensor 22, without considering the part related to time attenuation in the current response formula, so that more accurate results can be obtained.

In a preferred embodiment, the counter electrode 201 is arranged on a back side of the probe part 2, and two groups of working electrodes 202 and reference electrodes 203 are arranged on a front side of the probe part 2; compared with the traditional electrochemical sensor, the length of the counter electrode 201 is longer, and counter electrode 201 extends from the working sensor 21 to the associated sensor 22, covering the area where the two sensors are located.

The technical features of the above embodiments can be combined arbitrarily. To make the description concise, all possible combinations of the technical features of the above embodiments are not described. However, as long as there is no contradiction in the combination of these technical features, it should be considered to be within the scope of this specification.

The above embodiments only express some implementations of the present invention, and the descriptions thereof are relatively specific and detailed, but cannot be understood as limiting the scope of patent protection of the present invention. It should be noted that various modifications and improvements that can be made by those of ordinary skill in the art without departing from the concept of the present invention belong to the scope of protection of the present invention. Therefore, the scope of patent protection of the present invention shall be on the basis of the appended claims.

What is claimed is:

1. An electrochemical sensor based on an associated sensor, comprising an interface base and a probe part, wherein the interface base is provided with two groups of electrical connection terminals, the probe part is provided with a working sensor and an associated sensor, both sensors are provided with a counter electrode, a working electrode and a reference electrode, and the counter electrode of the working sensor and the counter electrode of the associated sensor are the same electrode;
one group of electrical connection terminals is correspondingly connected with the counter electrode, working electrode and reference electrode of the working sensor, and the other group of electrical connection terminals is correspondingly connected with the counter electrode, working electrode and reference electrode of the associated sensor.

2. The electrochemical sensor based on an associated sensor according to claim 1, wherein each group of electrical connection terminals comprises a first terminal, a second terminal and a third terminal, the first terminal is connected with the counter electrode, the second terminal is connected with the working electrode, and the third terminal is connected with the reference electrode.

3. The electrochemical sensor based on an associated sensor according to claim 1, wherein the associated sensor is arranged at an outer end of the probe part.

4. The electrochemical sensor based on an associated sensor according to claim 1, wherein the counter electrode is arranged on a back side of the probe part.

5. The electrochemical sensor based on an associated sensor according to claim 1, wherein the counter electrode extends from the working sensor to the associated sensor.
